(19) United States
(12) Patent Application Publication
(10) Pub. No.: US 2022/0280140 A1
(43) Pub. Date: Sep. 8, 2022

(54) PERSONAL HYGIENE SWABS

(71) Applicant: Frances Tang, Costa Mesa, CA (US)

(72) Inventor: Frances Tang, Costa Mesa, CA (US)

(21) Appl. No.: 17/637,995

(22) PCT Filed: Sep. 11, 2020
(86) PCT No.: PCT/US2020/050559

§ 371 Date: Feb. 24, 2022

Related U.S. Application Data
(60) Provisional application No. 62/899,869, filed on Sep. 13, 2019.

(51) Int. Cl. A61B 10/00

(52) U.S. Cl. CPC A61B 10/0045; A61B 2010/0074

(57) ABSTRACT

A personal hygiene swab includes a handle and an absorbent material secured to the handle. The handle is elongated and has a first end and a second end. The absorbent material includes an open-cell foam and covers a first end of the handle. The second end of the handle extends from the absorbent material. Another personal hygiene swab includes an absorbent material having a central cavity defining an opening at one end of the absorbent material. The absorbent material is stretchable, and a diameter of the central cavity is configured to accept a finger of a user. Removing fluid from a bodily cavity includes inserting a swab through a bodily orifice and into the bodily cavity, rotating the swab in the bodily cavity to absorb fluid in the bodily cavity, and removing the swab from the bodily cavity through the bodily orifice.

### CROSS-REFERENCE TO RELATED APPLICATION

This application claims priority to U.S. Application No. 62/899,869, filed Sep. 13, 2019, entitled PERSONAL HYGIENE DEVICE AND METHOD OF USE THEREOF, the disclosure of which is incorporated herein by reference.

### TECHNICAL FIELD

This invention relates to personal hygiene swabs for removing fluids from bodily cavities such as the vagina and anal cavity, including vaginal and anal walls and canals.

### BACKGROUND

Excess fluids from a variety of sources can be present in bodily cavities (e.g., the vagina and anal canal). In some cases, these excess fluids are bodily fluids. These excess fluids can be deposited in bodily cavities through bodily orifices by a variety of methods, including sexual intercourse. Examples of these excess fluids include ejaculate, personal lubricant, and vaginal fluids.

### SUMMARY

Inventive aspects described in this disclosure involve a realization that improvements in removing excess fluids from bodily cavities can promote the comfort and health of users. Conventional methods of removing excess bodily fluids from bodily cavities include wiping (e.g., with towels, wipes, tissues, toilet paper, clothing) and showering. These methods can provide incomplete removal, and can be inconvenient and inefficient. These methods can also be problematic, leading to an increased tendency to develop yeast and urinary infections and altering the natural pH in bodily orifices. Excess fluids in bodily orifices can also lead to dripping, odor, and stained clothing and bedsheets. These excess fluids can be uncomfortable, and can make sleeping and working inconvenient.

Various aspects of the disclosed personal hygiene swabs can effectively help a user remove excess fluids from bodily cavities quickly, safely, conveniently, comfortably, and thoroughly. Such aspects can include an absorbent material shaped and sized to absorb and remove excess fluids from bodily cavities without excessive drying. Some implementations include an extension or handle to facilitate inserting, maneuvering, and removing the absorbent material from the bodily cavity. Personal hygiene swabs described here are designed to remove fluids (e.g., bodily fluids) deposited in bodily cavities (e.g., vagina and anal canal) through bodily orifices (e.g., vaginal and anal orifices) during sexual intercourse or other sexual activities, mucous discharged from the cervix (including fluids from cervical creases that migrate down the vaginal canal), fluids released during menstruation, substances (e.g., lubricant) provided to bodily orifices, and the like.

In a first general aspect, a personal hygiene swab includes a handle and an absorbent material secured to the handle. The handle is elongated and has a first end and a second end. The absorbent material includes an open-cell foam and covers a first end of the handle. The second end of the handle extends from the absorbent material.

Implementations of the first general aspect include one or more of the following features.

In some implementations, the absorbent material surrounds at least half of the length of the handle. In some cases, the absorbent material is cylindrical or tubular. In some cases, the cross section of the absorbent material normal to a longitudinal axis of the handle is hexagonal. The absorbent material can have grooves, ridges, or both. The handle is typically elongated, and can be cylindrical or tubular. In certain cases, a cross section of the handle along its length is nonuniform. The handle can be retractable (e.g., telescoping). In some cases, the handle includes polypropylene. In certain cases, the second end of the handle includes a gripping portion (e.g., a spherical, “drop-shaped,” or cylindrical knob). The absorbent material can be heat bonded or adhered (e.g., with an adhesive) to the handle.

In a second general aspect, a personal hygiene swab includes an absorbent material having a central cavity and defining an opening at one end of the absorbent material. The absorbent material is stretchable, and a diameter of the central cavity is configured (e.g., sized and shaped) to accept a finger of a user.

Implementations of the second general aspect include one or more of the following features.

The central cavity typically extends along at least half of a length of the absorbent material. In some cases, the personal hygiene swab of the second general aspect includes a lip surrounding the opening. In certain cases, the personal hygiene swab include a handle (e.g., a flexible loop) extending from the absorbent material proximate the opening.

Implementations of the second general aspect may include one or more features of implementations of the first general aspect, as appropriate.

In a third general aspect, removing fluid from a bodily cavity includes inserting a swab through a bodily orifice and into the bodily cavity, rotating the swab in the bodily cavity to absorb fluid in the bodily cavity, and removing the swab from the bodily cavity through the bodily orifice.

Implementations of the third general aspect include one or more of the following features.

Removing fluid from the bodily cavity may include extending a handle of the swab (e.g., the personal hygiene swab of the first general aspect) before inserting the swab or positioning a finger in a cavity defined by the swab (e.g., the personal hygiene swab of the second general aspect) before inserting the swab. Rotating the swab typically includes rotating the finger positioned in the cavity. In some cases, implementations of the third general aspect include bending the finger positioned in the cavity to contact a portion of the bodily cavity with the swab. Removing the swab from the bodily cavity may include pulling a loop coupled to a base of the swab and extending from the bodily orifice. Implementations of the third general aspect typically includes removing, with the swab, fluid from an exterior bodily surface surrounding the bodily orifice.

The bodily cavity can be a vagina or an anal canal. The bodily fluid can be bodily fluid of the user (e.g. blood), bodily fluid from another person (e.g., ejaculate), or synthetic or naturally occurring fluid from an external source.

Advantages of personal hygiene swabs described herein include safe, comfortable, effective, efficient, convenient removal of fluid from a bodily cavity without drying the bodily cavity. These personal hygiene swabs are flexible, light, tear resistant, and biocompatible. They can be disposable or reusable (e.g., after rinsing or washing).

Before explaining the various aspects and implementations of the invention in detail, it is to be understood that aspects of the invention are not limited in its application to the details of construction and to the arrangements of the components set forth in the following description or illustrated in the drawings. Rather, aspects of the invention may include additional aspects and implementations. Also, it is to be understood that the terminology employed herein is for the purpose of description and should not be regarded as limiting.

The details of one or more implementations of the subject matter of this disclosure are set forth in the accompanying drawings and the description. Other features, aspects, and advantages of the subject matter will become apparent from the description, the drawings, and the claims.

### BRIEF DESCRIPTION OF DRAWINGS

FIG. 1 depicts an implementation of a personal hygiene swab having a handle.

FIG. 2 depicts an implementation of a personal hygiene swab having a handle.

FIG. 3 depicts an implementation of a personal hygiene swab having a telescoping handle with a grip.

FIG. 4 is a longitudinal cross-sectional view of an implementation of a personal hygiene swab with a cavity for finger insertion.

FIG. 5 is a perspective view of an implementation of a personal hygiene swab with an absorbent lip to facilitate external cleaning.

FIG. 6 is a longitudinal cross-sectional view of an implementation of a personal hygiene swab having a cavity for finger insertion and a loop for removal.

Like reference symbols in the various figures indicate like elements.

### DETAILED DESCRIPTION

Personal hygiene swabs for bodily orifices are described in this disclosure. These personal hygiene swabs are configured to be inserted directly into bodily orifices. Various aspects of the described personal hygiene swabs can effectively help a user remove excess fluids from bodily cavities. Such aspects can include an absorbent material shaped and sized to absorb and remove excess fluids from bodily cavities without excessive drying. Some implementations include an extension, handle, or cavity to facilitate inserting, maneuvering, and removing the absorbent material from the bodily cavity through the corresponding bodily orifice. These personal hygiene swabs are designed to remove fluids (e.g., bodily fluids deposited in bodily cavities, including vaginal and anal canals and walls, through vaginal and anal orifices) during sexual intercourse or other sexual activities, mucous discharged from the cervix (including fluids from cervical creases that migrate down the vaginal canal), fluids released during menstruation, substances (e.g., lubricant), and the like, through bodily orifices.

Absorbent material of the various implementations is a medical grade material that is tear-resistant and biocompatible. The absorbent material has open-cell pores sufficient to absorb and retain fluid (e.g., viscous fluid such as semen) without drying walls of a bodily cavity into which the absorbent material is inserted. In one example, the absorbent material is an open-cell foam. The absorbent material is typically selected to absorb at least two, three, or four times its weight in fluid. The absorbent material is flexible and light-weight, facilitating ease of use and maneuverability. In some cases, the absorbent material is elastic or stretchable. Suitable absorbent materials include polyurethane or other material (e.g., in the form of a foam or sponge) safe for insertion into a bodily orifice (e.g., sterile). A density of the absorbent material is typically in a range of 10 to 30 kg/m³.

The absorbent material is shaped and sized to fit comfortably through bodily orifices and into corresponding bodily cavities. In one example, a personal hygiene swab is designed to comfortably fit the shape and size of the vaginal canal during various phases of cyclical swelling. A length of the absorbent material is typically in a range of about 5 cm to about 7 cm (e.g., about 6 cm). A width or diameter of the absorbent material is typically in a range of about 1 cm to about 3 cm (e.g., about 2 cm). A cross-sectional shape of the absorbent material normal to a central axis along the length of the absorbent material can be a geometric shape (e.g., a circle, ellipse, oval, quatrefoil, triangle, curvilinear triangle, square, rectangle, trapezoid, rhombus, crescent, etc.). The size and shape of the absorbent material can be selected to achieve optimal absorption in a selected bodily cavity. In some cases, the absorbent material has groves, ridges, or both along a length of its exterior to promote absorption of fluids. In some cases, a length of the absorbent material is adjustable (e.g., retractable). The end of the absorbent material intended for insertion into a bodily cavity through a bodily orifice can be planar or have a rounded (convex) surface.

A personal hygiene swab can include a handle or extension or define a cavity to facilitate insertion into a bodily orifice, cleaning of a bodily orifice, and removal from a bodily orifice.

In some implementations, the absorbent material of a personal hygiene swab defines a cavity shaped and sized to comfortably accept a finger (e.g., an index finger) of a user. The cavity is typically a central cavity along a length of the absorbent material. An end of the absorbent material or an end of the cavity opposite its opening be rounded (e.g., concave). The absorbent material can be flexible and elastic. The user can bend the absorbent material by bending a finger inserted in the cavity. The opening is sized and shaped such that the swab remains securely on the finger of the user until the swab is removed by the user. The cavity can extend through about 50% to about 95% (e.g., about 75% to about 95%, or about 75% to about 85%) of the length of the absorbent material. In some cases, a personal hygiene swab with a cavity includes a portion (e.g., a string or a loop) near an opening of the cavity to facilitate removal of the personal hygiene swab from the bodily cavity. In certain cases, a personal hygiene swab with a cavity includes a lip (e.g., a radial extension) around an opening of the cavity to facilitate cleansing of an exterior portion of the body surrounding the bodily orifice. The lip can extend by about 25% to about 50% of the length of the absorbent material. A personal hygiene swab with a lip can be molded or shaped from a single piece of absorbent material.

In some implementations, a personal hygiene swab includes a handle. The handle is typically made of plastic (e.g., polypropylene). In some cases, the handle is flexible. The handle can be a solid elongated member having a length and diameter selected to facilitate insertion into a bodily cavity through a bodily orifice, cleaning of a bodily cavity, and removal from a bodily cavity through a corresponding bodily orifice. In some cases, the handle defines an interior opening along at least a part of its length (i.e., along a longitudinal axis of the handle). The handle can have a uniform cross section along and normal to its length. In some cases, the cross section along and normal to the length of the handle is circular (i.e., the handle is cylindrical or tubular). In some cases, the cross section is elliptical or lobed (e.g., quatrefoil). In certain cases, the handle has a nonuniform (e.g., ergonomic) cross section along and normal to its length. In some implementations, a diameter of the handle is in a range of about 0.3 cm to about 0.5 cm (e.g., about 0.4 cm). An exposed end of the handle can be texturized (e.g., with cross-hatching or protuberances) to facilitate gripping. In some implementations, the exposed end of the handle can have a shape or size selected to facilitate gripping. In one example, the exposed end of the handle includes a knob (e.g., a spherical or cylindrical knob) to facilitate gripping.

The absorbent material covers an end of the handle and surrounds a portion of the length of the handle. The handle extends from an end of the absorbent material sufficiently to allow comfortable gripping and use. In some implementations, the handle extends from an end of the absorbent material by about 5 cm to about 7 cm (e.g., about 6 cm). The handle can extend into the absorbent material by about 45% to about 95% of the length of the absorbent material (e.g., about 50% to about 95%, about 60% to about 95%, about 70% to about 95%, about 80% to about 95%, or about 90% to about 95%). The tip of the handle is typically embedded in the absorbent material, such that the tip of the handle does not contact a surface in the bodily cavity. In one example, the absorbent material has a length of 6 cm, and the handle extends into the absorbent material by about 3.5 cm to about 5.5 cm (e.g., about 4.5 cm to about 5.5 cm, or about 5.5 cm). A length of the personal hygiene swab typically exceeds a length of the handle.

In some cases, the handle is configured as a telescoping extension to facilitate compact packaging of the personal hygiene swab. In one example, a personal hygiene swab has a collapsible or retractable handle with a knob, such that the handle can be collapsed within the absorbent material before and after use, with only the knob exposed. The user can grip the knob and pull the handle away from the absorbent material, thereby extending the handle for use.

The handle is coupled to an interior surface of the absorbent material. In one example, the handle is bonded to an interior of the absorbent material. The absorbent material can be securely attached to and surround a circumference and a tip of the handle, thereby preventing the tip of the handle from contacting an interior surface of the bodily cavity. Bonding can include heat bonding or bonding with an adhesive or other appropriate method to prevent detachment of the handle from the absorbent material during use.

In some implementations, a personal hygiene swab is washable and reusable. In one example, a personal hygiene swab can be rinsed and dried for reuse. In some implementations, a personal hygiene swab is biodegradable, flushable, collapsible, or any combination thereof.

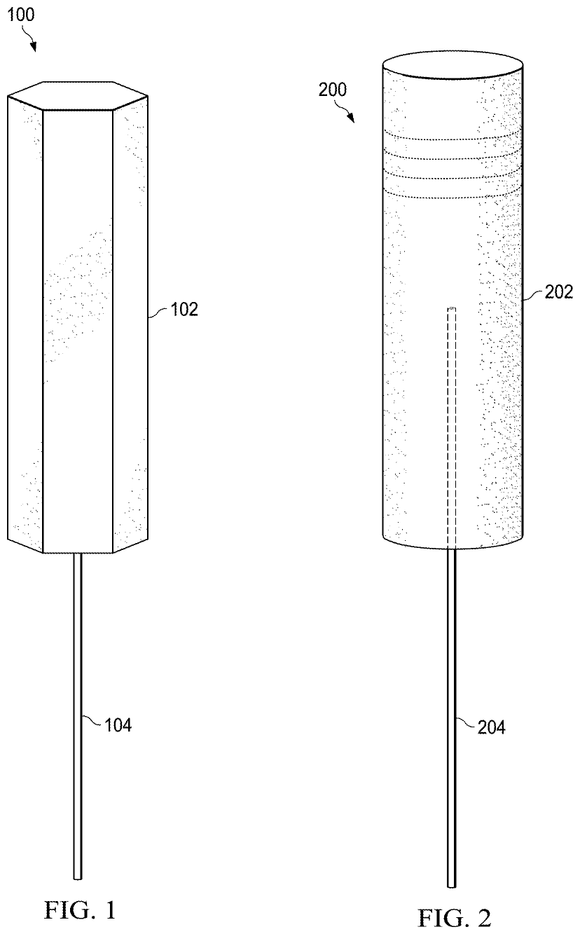

FIG. 1 depicts personal hygiene swab 100. Personal hygiene swab 100 includes absorbent material 102 and handle 104 as described herein. Absorbent material 102 has a hexagonal cross section normal to a longitudinal axis of the handle.

FIG. 2 depicts personal hygiene swab 200. Personal hygiene swab 200 includes absorbent material 202 and handle 204 as described herein. Handle 204 extends about halfway into absorbent material 202. Absorbent material 202 is cylindrical in shape, with a circular cross section normal to a longitudinal axis of the handle.

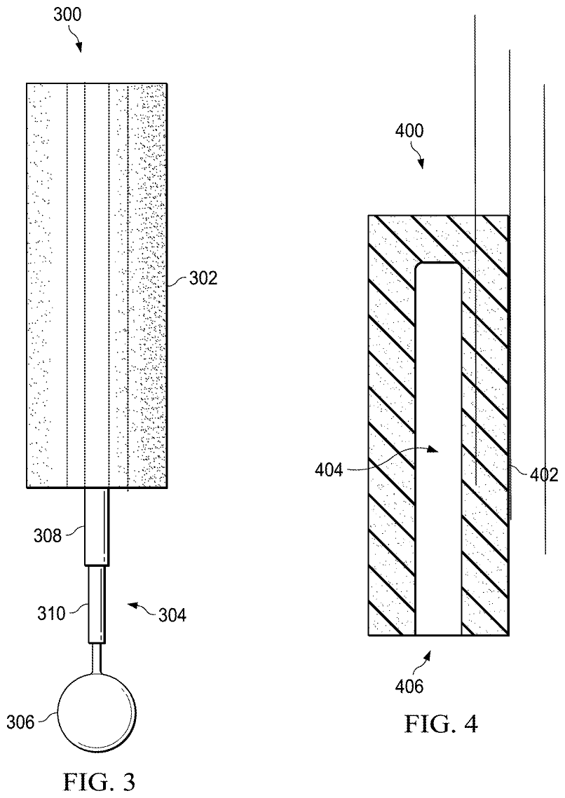

FIG. 3 depicts personal hygiene swab 300. Personal hygiene swab 300 includes absorbent material 302 as described herein. Personal hygiene swab 300 also includes telescoping handle 304 with grip portion 306 and having properties as described herein. As depicted, grip portion 306 is spherical. In other implementations, grip portion 306 can have a cylindrical shape, a hexagonal shape, an oval or “drip” shape, or other shape suitable for gripping. Outer section 308 has an inner diameter that exceeds the outer diameter of inner section(s) 310 of handle 304. Grip portion 306 can be advanced toward absorbent material 302 to translate inner section(s) 310 of handle 304 through the inner diameter of outer section 308, thereby reducing a length of handle 304 extending beyond absorbent material 302. In some cases, with handle 304 collapsed inside absorbent material 302, only grip portion 306 of handle 304 is visible at an end of absorbent material 302.

FIG. 4 is a longitudinal cross-sectional view of personal hygiene swab 400. Personal hygiene swab 400 includes absorbent material 402 as described herein. Absorbent material 402 defines cavity 404 with opening 406 as described herein for insertion of a user's finger. Cavity 404 allows a user to control movement of personal hygiene swab 400 to effectively absorb excess fluid from and cleanse the bodily cavity into which it has been inserted.

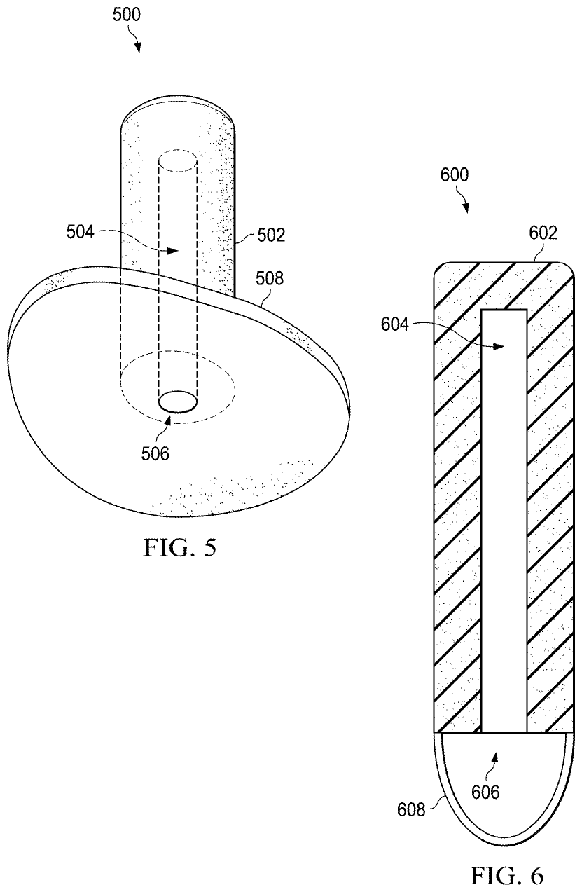

FIG. 5 is a perspective view of personal hygiene swab 500. Personal hygiene swab 500 includes absorbent material 502 as described herein. Absorbent material 502 defines cavity 504 with opening 506 for insertion of a user's finger as described herein. Cavity 504 allows a user to control movement of personal hygiene swab 500 to effectively cleanse the bodily cavity into which it has been inserted. Absorbent material 502 includes lip 508. Lip 508 flares out or extends radially from absorbent material 502 surrounding opening 506. Lip 508 is configured to collect fluids outside the bodily orifice corresponding to the bodily cavity into which personal hygiene swab 500 has been inserted. In some cases, lip 508 is planar. In other cases, lip 508 is curved toward or away from the opening 506 of cavity 504. Lip 508 can cleanse a portion of skin surface surrounding the bodily orifice. In one example, lip 508 facilitates collection of fluids from and cleansing of the labia and outer vaginal area. In some implementations, personal hygiene swab 500, including lip 508, is formed or molded as a unitary piece.

FIG. 6 depicts personal hygiene swab 600. Personal hygiene swab 600 includes absorbent material 602 as described herein. Absorbent material defines cavity 604 with opening 606 as described herein. Personal hygiene swab 600 includes loop 608 proximate opening 606 of absorbent material 602. Loop 608 extends from the end of the personal hygiene swab 600. Loop 608 can be used (e.g., pulled) away from the bodily orifice) to facilitate removal of personal hygiene swab 600 from a bodily cavity in which it is positioned.

A personal hygiene swab can be used to collect excess fluids from a bodily cavity or orifice. Use of a personal hygiene swab having a handle includes gripping the grip portion or handle of the personal hygiene swab, inserting the swab into a bodily orifice, rotating the swab in the bodily cavity to absorb excess fluids, and removing the fluid-laden swab from the bodily cavity through the corresponding bodily orifice using the handle. Use of a personal hygiene swab defining a cavity for finger insertion includes inserting a finger into the cavity, inserting the swab into a bodily cavity through the corresponding bodily orifice, rotating the swab in the bodily cavity to absorb excess fluids, and removing the fluid-laden swab from the bodily cavity through the bodily orifice through which it was inserted. These processes can be repeated as appropriate until a desired amount of fluid has been removed from the bodily cavity.

Although this disclosure contains many specific embodiment details, these should not be construed as limitations on the scope of the subject matter or on the scope of what may be claimed, but rather as descriptions of features that may be specific to particular embodiments. Certain features that are described in this disclosure in the context of separate embodiments can also be implemented, in combination, in a single embodiment. Conversely, various features that are described in the context of a single embodiment can also be implemented in multiple embodiments, separately, or in any suitable sub-combination. Moreover, although previously described features may be described as acting in certain combinations and even initially claimed as such, one or more features from a claimed combination can, in some cases, be excised from the combination, and the claimed combination may be directed to a sub-combination or variation of a sub-combination.

Particular embodiments of the subject matter have been described. Other embodiments, alterations, and permutations of the described embodiments are within the scope of the following claims as will be apparent to those skilled in the art. While operations are depicted in the drawings or claims in a particular order, this should not be understood as requiring that such operations be performed in the particular order shown or in sequential order, or that all illustrated operations be performed (some operations may be considered optional), to achieve desirable results.

Accordingly, the previously described example embodiments do not define or constrain this disclosure. Other changes, substitutions, and alterations are also possible without departing from the spirit and scope of this disclosure.

1. A personal hygiene swab comprising:
a handle, wherein the handle is elongated and comprises a first end and a second end; and
an absorbent material secured to the handle and comprising an open-cell foam,
wherein the absorbent material covers a first end of the handle, and the second end of the handle extends from the absorbent material.

2. The swab of claim 1, wherein the absorbent material surrounds at least half of the length of the handle.

3. (canceled)

4. (canceled)

6. The swab of claim 1, wherein a length of absorbent material is adjustable.

7. The swab of claim 1, wherein the absorbent material comprises grooves, ridges, or both.

8. (canceled)

9. (canceled)

10. The swab of claim 1, wherein a cross section of the handle along its length is nonuniform.

11. The swab of claim 1, wherein the handle is retractable.

12. (canceled)

13. The swab of claim 1, wherein the second end of the handle comprises a gripping portion in the shape of a spherical knob.

14. (canceled)

15. (canceled)

16. A personal hygiene swab comprising:
an absorbent material having a central cavity defining an opening at one end of the absorbent material, wherein the absorbent material is stretchable, and a diameter of the central cavity is configured to accept a finger of a user.

17. The swab of claim 16, wherein the central cavity extends along at least half of a length of the absorbent material.

18. The swab of claim 16, further comprising a lip surrounding the opening.

19. The swab of claim 16, further comprising a handle extending from the absorbent material proximate the opening.

20. The swab of claim 19, wherein the handle comprises a flexible loop.

21. A method for removing fluid from a vagina, the method comprising:
inserting a swab through a bodily orifice and into the bodily cavity;
rotating the swab in the bodily cavity to absorb fluid in the bodily cavity; and
removing the swab from the bodily cavity through the bodily orifice.

22. The method of claim 21, further comprising extending a handle of the swab before inserting the swab.

23. (canceled)

24. The method of claim 21, further comprising positioning a finger in a cavity defined by the swab before inserting the swab.

25. The method of claim 24, wherein rotating the swab comprises rotating the finger positioned in the cavity.

26. The method of claim 24, further comprising bending the finger positioned in the cavity to contact a portion of the bodily cavity with the swab.

27. The method of claim 21, wherein removing the swab from the bodily cavity comprises pulling a loop coupled to a base of the swab and extending from the bodily orifice.

28. (canceled)

29. The method of claim 21, wherein the fluid comprises bodily fluid from another person.

30. The method of claim 29, wherein the fluid comprises ejaculate.

31. (canceled)
